(19) United States
(12) Patent Application Publication
(10) Pub. No.: US 2024/0285906 A1
(43) Pub. Date: Aug. 29, 2024

(54) MEDICAL DEVICE CONTROL HANDLE WITH INTEGRATED FLUSH PORT/SEAL FEATURES

(71) Applicant: Cook Medical Technologies LLC, Bloomington, IN (US)

(72) Inventor: Palle Munk Hansen, Bjaeverskov (DK)

(73) Assignee: Cook Medical Technologies LLC, Bloomington, IN (US)

(21) Appl. No.: 18/411,803

(22) Filed: Jan. 12, 2024

Related U.S. Application Data
(60) Provisional application No. 63/439,057, filed on Jan. 13, 2023.

(51) Int. Cl. A61M 25/01; A61M 39/00

(52) U.S. Cl. CPC A61M 25/0136; A61M 39/00; A61M 2039/0018

(57) ABSTRACT

Described are systems for hand-held control of medical device structures. The systems can include a control handle including a handle housing and an actuator movably mounted to the handle and attached to a control shaft. A sheath is attached to a distal end of the handle housing and has a lumen fluidly communicating with a distal opening of the sheath. The control shaft extends from the handle housing and into the lumen of the sheath. A medical device structure is attached to the control shaft and the actuator is operable by the hand of the user to cause movement of the control shaft distally relative to the handle housing, for example so as to deploy the medical device structure from the distal opening of the sheath, and proximally relative to the handle housing, for example so as to retract the medical device structure into the distal opening of the sheath. A seal element is mounted in the handle housing and the control shaft extends through the seal element. The seal element is cooperable with the control shaft to maintain a seal against the control shaft during the movement of the control shaft. A flush port is provided which fluidly communicates with the lumen of the sheath and can also fluidly communicate with a surface of the seal element. Handles useful in such systems, as well as related methods of manufacture and use, are also described.

### REFERENCE TO RELATED APPLICATIONS

This application claims the benefit of priority of U.S. Provisional Application No. 63/439,057 filed Jan. 13, 2023 which is hereby incorporated by reference in its entirety.

### FIELD

Aspects of the present disclosure relate generally to medical devices. More particularly, certain aspects of the present disclosure relate to medical device control systems that have control handles with an actuator to manipulate a control shaft and attached medical device structure, and that incorporate a seal element associated with the control shaft and/or a flushing port for delivering a flushing medium into a lumen of a sheath associated with the control handles.

### BACKGROUND

The statements in this section merely provide background information related to the present disclosure and may not constitute prior art.

Medical device control systems often incorporate a handle for effective operation of components of the systems. A variety of operations are commonly desired during preparation and operation of such medical device systems, which can tend to make system structures, and their manufacture and use, somewhat complex. There remain needs for improved and alternative medical device control handles and associated medical device control systems, and methods for their manufacture and use. Desirably, the subject handles and systems will be effective and facile in use as well as practicable in manufacture. In at least some of its aspects, the present disclosure is addressed to such needs.

### SUMMARY

In one aspect, the present disclosure relates to a system for hand-held control of a medical device structure. The system includes a control handle including a handle housing and an actuator movably mounted to the handle housing and arranged for manual operation by a hand of a user. The system also includes a control shaft connected to the actuator and a sheath attached to a distal end of the handle housing. The sheath has a lumen extending to a distal opening of the sheath, and the control shaft extends from the handle housing and into the lumen of the sheath. A medical device structure is attached to the control shaft, and the actuator is operable by the hand of the user to cause movement of the control shaft distally relative to the handle housing and proximally relative to the handle housing. The movement of the control shaft distally relative to the handle housing can deploy the medical device structure from the distal opening of the sheath and the movement of the control shaft proximally relative to the handle housing can retract the medical device structure into the distal opening of the sheath. A seal element is mounted in the handle housing, with the control shaft extending through the seal element. The seal element is cooperable with the control shaft to maintain a seal against the control shaft during the movement of the control shaft. The system also includes a flush port opening in fluid communication with the lumen of the sheath. The flush port opening can be on the control handle. In some forms, the seal element can interrupt a fluid passage extending from the distal opening of the sheath, through the lumen of the sheath and into an interior space of the handle housing. The seal element, for example in the form of one or multiple seal discs, can include at least a first opening therein, with the control shaft received through the first opening and being longitudinally slidable and rotatable relative to the first opening.

In another aspect, the present disclosure relates to a method for preparing a system prior to use on a patient. The method includes providing a system as described above and/or elsewhere in the present disclosure, and passing a flush liquid into the flush port opening, through the lumen of the sheath, and out the distal opening of the sheath.

In still another aspect, the present disclosure relates to a method for treating a patient using a medical device. The method includes providing a system as described above and/or elsewhere in the present disclosure, and passing a flush liquid into the flush port, through the lumen of the sheath, and out the distal opening of the sheath. The method further includes, after the above-described step of passing a flush liquid, introducing the sheath into a vascular vessel of the patient, operating the actuator by hand to cause movement of the control shaft distally relative to the handle housing so as to deploy the medical device from the distal opening of the sheath, and treating the patient with the medical device.

In still another aspect, the present disclosure relates to a medical device control handle for hand-held control of a medical device structure that is connected to a control shaft and deployable from a lumen of a sheath. The medical device control handle includes a handle housing and an actuator movable relative to the handle housing and arranged for manual operation by a hand of a user, the actuator being connectable to the control shaft. The handle also includes at least one of, or both of: a) a seal assembly mounted in the handle housing and configured to maintain a seal against an outer surface of a control shaft for a medical device, the seal assembly including a seal body defining a lumen therein and an elastomeric seal element received against the seal body; and b) a flush port assembly mounted in the handle housing, the flush port assembly including a first flush port body portion defining a first flush lumen portion therein configured to fluidly communicate with a sheath lumen, a second flush port body portion extending transverse to the first flush port body portion and providing a flush port stem defining a second flush lumen portion therein, the second flush lumen portion intersecting and fluidly communicating with the first flush lumen portion. The seal assembly when included can also include a seal cap attached to the seal body so as to press the elastomeric seal element against the seal body portion. Handles that include both the seal assembly and the flush port assembly are preferred, and in such embodiments the flush port assembly and the seal assembly can be subcomponents of a flush port/seal assembly, beneficially where the seal body, the first flush port body portion, and the second flush port body portion are segments of an integral flush port/seal body. The handle housing can have one or more inwardly-directed walls that contact and positionally fix the flush port assembly or the seal assembly relative to the handle housing. The handle housing can be composed of at least, and in some forms only, a first handle housing portion and a second handle housing portion connected together.

Still further aspects of the present disclosure, as well as features and advantages thereof, will be apparent to those skilled in the art from the descriptions herein.

### BRIEF DESCRIPTION OF THE FIGURES

In order that the disclosure may be well understood, there will now be described various forms thereof, given by way of example, reference being made to the accompanying drawings. The components in each of the drawings may not necessarily be drawn to scale, rather emphasis is placed upon illustrating the principles of the invention. Moreover, like referenced numerals in different drawings designate corresponding or similar components or elements.

FIG. 1 provides a partial cutaway, perspective view of one embodiment of a medical device control system in accordance with the present disclosure.

FIG. 2 provides a partial cutaway top view of the medical device control system depicted in FIG. 1.

FIG. 3 provides a partial cutaway view of a distal region of a medical device control handle of the system depicted in FIG. 1, showing internal features of the handle.

FIG. 4 provides an exploded view of components of the distal region of the medical device control handle depicted in FIG. 3.

FIG. 5 provides a partial cutaway view showing of components of the system depicted in FIG. 1 in a partially disassembled state.

FIG. 6 provides a partial cutaway view of components of another embodiment of a medical device control system in accordance with the present disclosure.

### DETAILED DESCRIPTION

Reference will now be made to certain embodiments, some of which are illustrated in the drawings, and specific language will be used to describe the same. It will nevertheless be understood that no limitation of the scope of the disclosure herein is thereby intended. Any alterations and further modifications in the described embodiments and any further applications of the principles as described herein are contemplated as would normally occur to a person skilled in the art to which the embodiments relate.

As disclosed above, certain embodiments of the present disclosure relate to systems for hand-held control of a medical device structure, to handles and other components thereof, and to methods of their manufacture and use.

For the purpose of the present disclosure, the term “proximal” refers to a direction that is generally towards a user (e.g., physician) during a medical procedure, while the term “distal” refers to a direction that is generally towards a target site within a patient's anatomy during the medical procedure.

For the purpose of the present disclosure, the terms “at least one” and “one or more of” an element are used interchangeably and may have the same meaning. These terms, which refer to the inclusion of a single element or a plurality of the elements, may also be represented by the suffix “(s)” at the end of the element. For example, “at least one metal”, “one or more metals”, and “metal(s)” may be used interchangeably and are intended to have the same meaning.

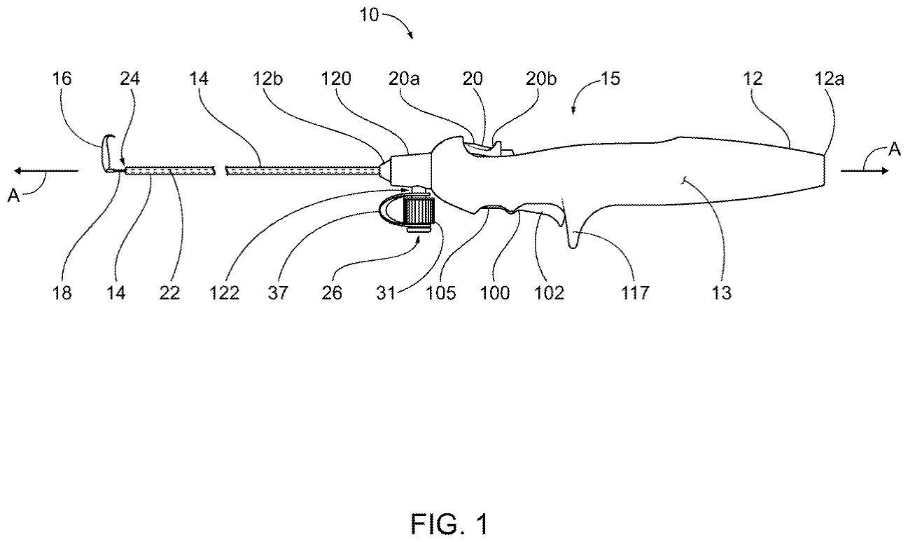

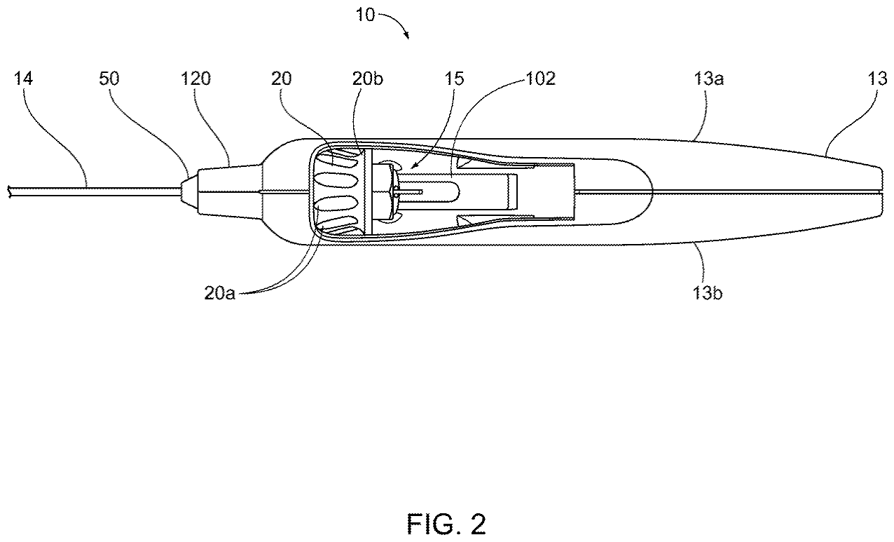

With reference now to FIGS. 1 and 2, shown are a partial cutaway, perspective view and a partial cutaway top view, respectively, of one embodiment of a medical device control system 10 in accordance with the present disclosure. System 10 generally includes a medical device control handle 12 having a proximal end 12a and distal end 12b to which is attached a sheath 14 (which can, for instance, be a catheter). System 10 includes a snare element, such as a vascular snare element, having a snare loop 16, although in other forms the snare element may include hooks or multiple loops, or the snare element may instead be another medical device structure, for example all of or a portion of another gripping member such as a multi-pronged clamp or clasp. The snare loop 16 or other medical device structure can be connected to a longitudinally extending control shaft 18, which in turn can be connected to an actuator such as an actuator button 20. Snare loop 16 and control shaft 18 can each be made of a single component or multiple components, can be made of any suitable material such as a polymeric and/or metal material, and can be or include solid cross-sectional (e.g. solid wire structures) or hollow parts (e.g. cannula structures), as known in the art. As well, snare loop 16 and control shaft 18 may be a single integral component. In some configurations, snare loop 16 is a braided wire loop.

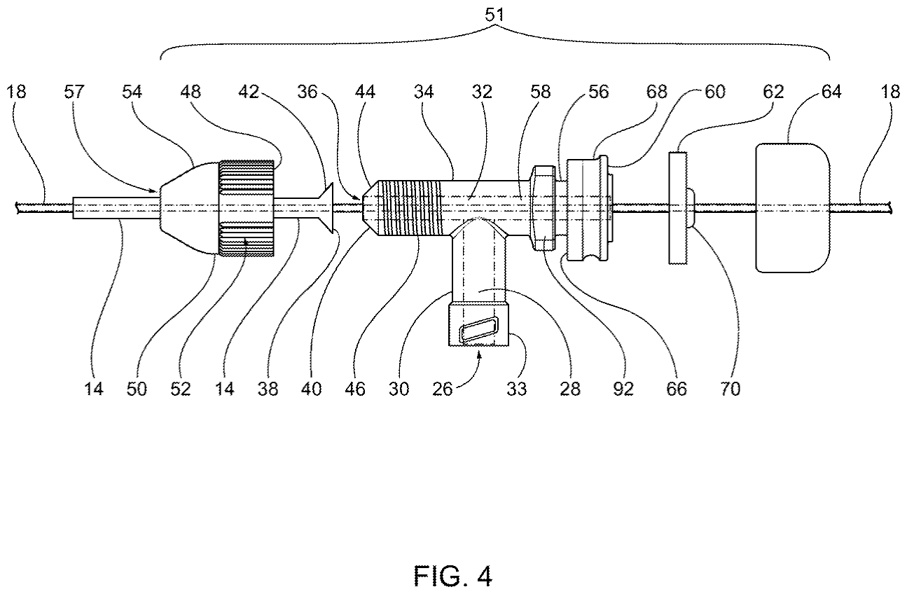

In some forms, the longitudinally extending control shaft 18 extends to directly connect with actuator button 20, for example being received and secured within actuator button, for example by a locking mechanism, an adhesive, or both, or other techniques. The medical device control handle 12 includes a handle housing 13 that defines a cutout 15 that creates a movement space therein. The actuator button 20 is disposed within the cutout 15 and connected to the control shaft 18. The actuator button 20 may be movable within the movement space defined by the cutout 15 along the longitudinal axis (A-A) of the system 10 and/or rotatable within the movement space defined by the cutout 15, for example with a rotational axis also along the longitudinal axis A-A. The actuator button 20 can be movable, for example slidable, along the longitudinal axis A-A independent of rotation of the actuator button 20. The longitudinal movement of the actuator button 20 can correspondingly longitudinally move the control shaft 18 and the snare loop 16 or other medical device structure attached to control shaft 18, and/or the rotation of the actuator button 20 can correspondingly rotate the control shaft 18 and the snare loop 16 or other medical device structure attached to the control shaft 18. The longitudinal movement and/or rotational movement associated with the actuator button 20 may be incorporated such that one finger of the user is capable of controlling both the longitudinal and/or rotational movement of the medical device by a sliding motion and/or a scrolling motion, respectively. In some forms, the actuator button 20 may be a thumb wheel capable of being manipulated solely by the thumb finger of the user (e.g., physician) in order to move the control shaft 18 and the snare loop 16 (or other medical device structure) both longitudinally and rotationally, for example through the lumen 22 of sheath 14 and/or within the patient when deployed out of the end opening 24 of sheath 14. When desirable, the thumb wheel or other actuator button 20 as shown for example in FIGS. 2 and 4 may include one or more fins 20a and/or an indented edge 20b configured to fit a thumb finger to provide for additional gripping during rotational and longitudinal movements. When the actuator button 20 is a thumb wheel the fins 20a may be disposed around the circumference of the wheel, and/or the button 20 can define a generally cylindrical exterior profile.

The handle includes a handle housing 13 that may be a singular unified component or include a plurality of components, such as an upper body portion and a lower body portion, or such as a left-side body portion and a right-side body portion, that are attached together by any suitable mechanism, including for example by correspondingly-located pins and holes for receiving the pins, gluing, welding, melt bonding, combinations thereof, or the like. The dimensions of the handle housing 13 may be customized in order to fit a specific predetermined hand size or be optimized such that various hand sizes are capable of obtaining a firm grip when using the handle 12. Additional ergonomic features may be included in the elongate handle housing as well without exceeding the scope of the present disclosure.

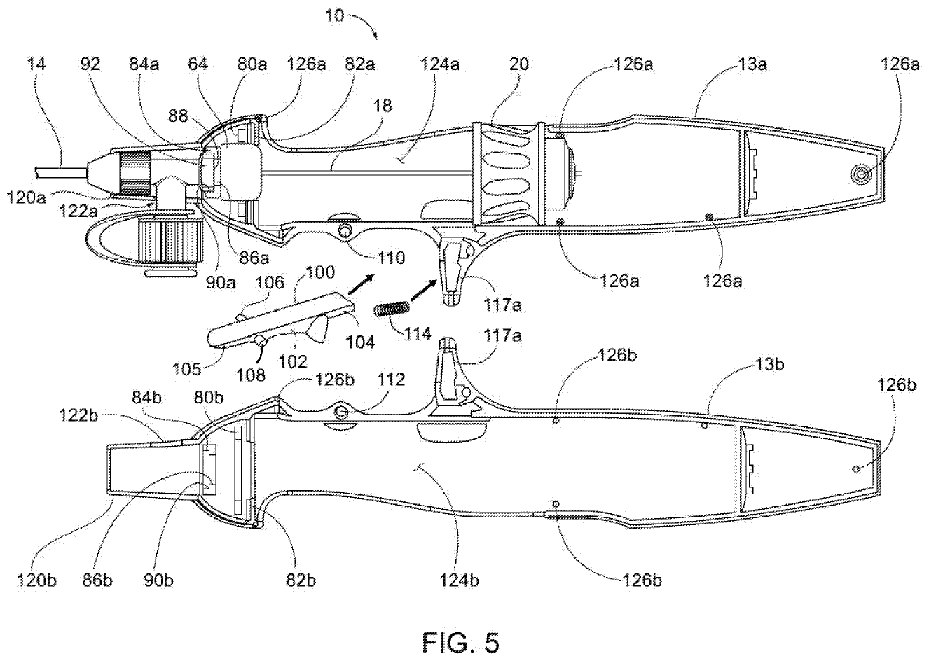

The control shaft 18 in the illustrated embodiment extends through the lumen 22 of sheath 14 and into the handle 12. When, as illustrated, the actuator button 20 is in its forward (distally-positioned) position, the snare loop 16 is deployed from the distal opening 24 of the sheath 14. When the actuator button 20 is in its rearward (proximally-positioned) position (see e.g. FIG. 5), the snare loop 16 is positioned within the lumen 22 of sheath 14. Thus, button 20 can be translated distally and proximally to deploy and retract the snare loop 16, respectively, from and into the lumen 22 of sheath 14. The actuator button 20 can also be rotated to rotate the snare loop 16, for example after the snare loop 16 is deployed distally out of the distal opening 24 of sheath 14.

In some forms, as illustrated, the snare loop 16 extends at an angle relative to the longitudinal axis of the control shaft 18 when deployed distally out of the distal opening 24 of sheath 14, and the actuator button 20 can be rotated to rotate the angled snare loop 16 to different radially positioned capture orientations. In use, this can facilitate positioning the snare loop 16 around a portion of an implanted medical device (which can be a vascular filter, such as a vena cava filter) to be retrieved, after which the actuator button 20 can be moved proximally to draw the snare loop 16 partially into the lumen 22 of sheath 14 so as to tighten the snare loop 16 around the implanted medical device. In some forms, the implanted medical device can be drawn into the lumen 22 of sheath 14 for removal from the patient. In other forms, the system 10 may be used in conjunction with another sheath or catheter, and the system 10 may be used to draw the implanted medical device into the other sheath or catheter. These and other modes of use for system 10 may be employed.

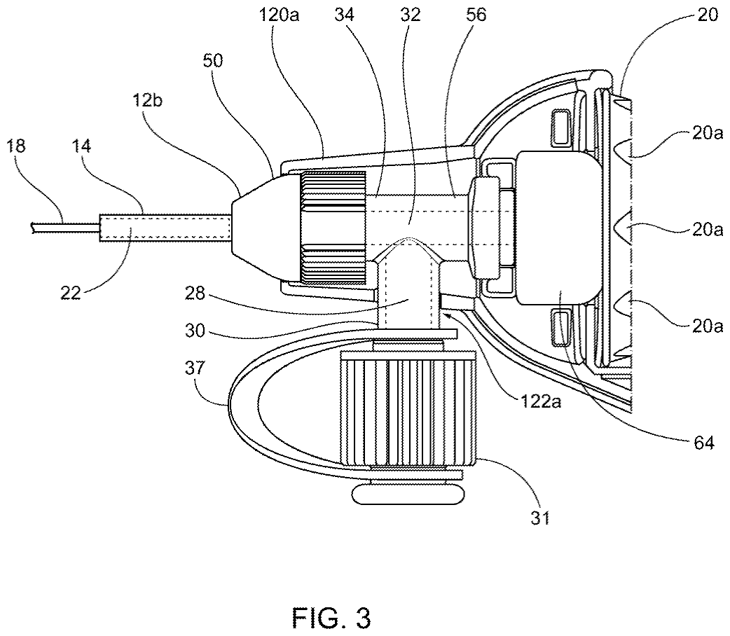

With continued reference to FIGS. 1 and 2 now also in connection with FIGS. 3 and 4, aspects of the system 10 relating to a flush port and/or seal arrangement will now be described. The illustrated control handle 12 incorporates a flush port opening 26 on the handle 12 that fluidly communicates with the lumen 22 of sheath 14. In the embodiment shown, prior to use of the system 10 on a patient, the flush port opening 26 can be utilized to introduce a flush liquid, such as water or saline, to pass into and distally through lumen 22 and exit distal opening 24 of sheath 14, for example to flush air from the lumen 22 (and in particular to flush the generally annular space between the outer surface of the control shaft 18 and the inner surface of sheath 14 that defines the lumen 22). The flush port opening 26 communicates with a flush port lumen 28 defined by a flush port stem 30, with the flush port lumen 28 and stem 30 extending transversely (e.g. perpendicularly) to the longitudinal axis A-A of the control handle 12 and/or system 10. The flush port lumen 28 intersects and fluidly communicates with a longitudinal flush lumen 32 defined by a longitudinally-extending flush body portion 34. The longitudinal flush lumen 32 in turn fluidly communicates with the lumen 22 of sheath 14, so as to direct flush liquid introduced through flush port opening 26 into the lumen 22 of sheath 14. The longitudinal flush lumen 32 also fluidly communicates with a surface of a seal element 62, to be discussed further below. In the illustrated embodiment, the longitudinally-extending flush body portion 34 defines a distal opening 36 of the longitudinal flush lumen 32 that opens into the sheath lumen 22. This can be provided by a connection of the proximal end 38 of the sheath 14 to the distal end 40 of the longitudinally-extending flush body portion 34. The embodiment shown includes an outwardly flared proximal end region 42 of the sheath 14 that mates with a distally tapered region 44 of the longitudinal flush body portion 34. As well, the outer surface of the longitudinally-extending flush body portion 34 defines a threaded region 46 (see in particular FIG. 4) that threadably meshes with a threaded region 48 on the interior surface of the connector cap 50. Connector cap 50 can thereby be threaded onto the longitudinally-extending flush body portion 34 using threaded regions 46 and 48 to threadably connect the connector cap 50 and flush body portion 34, and in so doing the connector cap 50 can press the flared proximal end region 42 of sheath 14 against the distally tapered region 44 of flush body portion 34 to create a seal between them. The connector cap 50 can be sized and include inner surfaces contoured to achieve such a seal. The connector cap 50 may also include longitudinally-extending grip ridges 52 on its external surface to aid in threading operations during assembly and/or a distally-tapering distal region 54 that can define inner surfaces contoured to engage and press the flared proximal end region 42 to create the seal as discussed above when connector cap 50 is threaded onto flush body portion 34. The connector cap 50 also defines a distal cap opening 57 through which sheath 14 extends. A flush port cap 31 can also be provided that is removably attachable to the flush port stem 30 to cover and/or seal the flush port opening 26, and in such cases a strap 37 can also be provided that connects the flush port cap 31 to the flush port stem 30. For these purposes external surfaces of the flush port stem 30 can define a threaded luer or other threaded structure 33 (see FIG. 4) and internal surfaces of the cap 31 can define a corresponding threaded structure (not shown) for threaded attachment of the cap 31 to the stem 30. The flush port cap 31 can be removed from flush port stem 30 to enable a flushing operation as described herein and/or can be attached to the flush port stem 30 (e.g. re-attached after such removal and flushing) so as to seal flush port opening 26, which during use of the system 10 to treat a patient can also prevent any bodily liquid (e.g. blood) that backflushes proximally through lumen 22 of sheath 14 and into the flush port lumen 28 from exiting the flush port opening 26 and/or prevent the entry of air into the flush port opening 26.

The illustrative system 10 also includes a sealing arrangement to provide a seal against control shaft 18, including during movement of control shaft 18 caused by movement of the actuator button 20. Such seal can serve to prevent backflushing of patient bodily fluid (e.g. patient blood) proximally through the lumen 22 of sheath 14 and into an interior space of the medical device control handle 12, for example an interior space from which the bodily fluid can leak out of an opening of the handle housing 13. In the illustrated embodiment, this sealing arrangement is advantageously provided by structures integrally connected to the flush port opening 26 and its associated components as discussed above, although this is not necessary in other embodiments. Particularly, as illustrated, a proximal seal body portion 56 is connected to the flush body portion 34, and can in a beneficial embodiment be formed as an integral, monolithic piece with the flush body portion 34 and the flush port stem 30. The proximal seal body portion 56 defines a proximal seal body portion lumen 58 that fluidly communicates with the longitudinal flush lumen 32 and that can be axially aligned therewith.

Proximal seal body portion 56 defines a proximal end surface 60 against which is received a seal element 62, for example in the form of a seal disc as illustrated. The seal element can sealingly interrupt a fluid passage extending from the distal opening 24 of the sheath 14, through the lumen 22 of the sheath 14 and into an interior space of the handle housing 13, for example such interior space being a movement space in which actuator 20 moves longitudinally and/or rotates as in the illustrated embodiment. Such fluid passage can in some forms also include a lumen defined by a flush body (e.g. flush body portion 34 and its lumen 32) and/or a lumen defined by a seal body (e.g. proximal seal body portion 56 and its lumen 58). The seal disc or other seal element 62 can be formed from a resilient material, for example an elastomeric material, that can conform to the outer surface of control shaft 18. For example, the seal disc or other seal element 62 can be formed from a polymeric elastomer such as silicone or rubber. The seal disc or other seal element 62 can provide a seal barrier that extends across the seal body lumen 58. The seal disc or other seal element 62 can be pressed (and potentially compressed) against the proximal end surface 60 by a seal cap 64 attached to the proximal seal body portion 56, for example by press fitting. For these purposes, the proximal seal body portion 56 can define a shoulder 66 and/or groove 68 that can cooperate with interior surfaces of the seal cap 64 to secure the seal cap 64 to proximal seal body portion 56 when press fit thereon. In certain embodiments, a seal element 62 in the form of a seal disc can define an annular ring portion 70 that extends proximally from adjacent surfaces of the seal disc and that surrounds an inner portion of the seal disc through which the control shaft 18 passes in the assembled system 10, with such inner portion forming a seal against the outer surface of the control shaft 18 as discussed herein. As shown for example in FIG. 4, a portion of the control shaft 18 can be suspended in the handle housing 13 between the actuator 20 and the seal element 62, and it will be understood that the portion of the control shaft 18 so suspended can shorten as actuator 20 is moved distally and lengthen as actuator 20 is moved proximally. In some embodiments, the seal element 62, for example a sealing disc, will define a slit, multiple slits, a weakened portion such as a score, and/or multiple such weakened portions, to provide or facilitate the creation of an opening in the seal element 62 through which control shaft 18 extends in the system 10. In some forms, a single seal disc or other seal element 62 can be provided through which control shaft 18 extends. In other forms, multiple discrete seal discs or other seal elements 62 can be provided through which control shaft 18 extends, for example with such seal discs or other seal elements being in a configuration stacked against one another. These and other seal arrangements will be apparent to those skilled in the field and may be used within handles and systems of the present disclosure.

The control shaft 18 can be frictionally translated proximally and distally through and against seal element 62 and/or can be frictionally rotated within and against seal element 62, while maintaining a seal between the outer surface of control shaft 18 and the proximal seal body portion 56. In this manner, any bodily fluid, for example blood, that passes into the lumen 22 of the sheath 14 from its distal opening 24 (e.g. in the annular space between the control shaft 18 and the surface defining the lumen 22 of the sheath 14), is prevented from passing proximally past the seal element 62 and into an interior space of the medical device control handle 12. Also, when a flush liquid is introduced into flush port opening 26 under pressure, the flush liquid may pass through flush port lumen 28 and into proximal seal body portion lumen 58 and against a surface of the seal element 62. In such cases the seal element 62 can also prevent flush liquid from passing proximally past the seal element 62 and into interior spaces of the medical device control handle 12. As well, the friction between the control shaft 18 and seal element 62 can facilitate a smoother and more tactile operation of the actuator button 20 as it is translated distally and proximally by a user and/or is rotated by a user.

While medical snare embodiments above have sometimes been described having a single snare loop 16, it will be understood that in other embodiments other medical device structures, for example other snare capture elements that may include multiple snare loops or hooks, or all or part of another gripping member such as a clamp or clasp with multiple prongs (e.g. that may be urged toward one another by retracting a part of the gripping member into lumen 22 of sheath 14) may be used in the place of the snare loop 16. Such other medical device structures can be connected to the control shaft 18 and can be deployed from and retracted into the lumen 22 of the sheath 14 using movement of the actuator button 20 along the longitudinal axis A-A and/or can be rotated by rotating the actuator button 20, as described above.

Referring now also to FIG. 5 in conjunction with FIGS. 1-4, shown in FIG. 5 is a partial cutaway view of components of the system 10 in a partially disassembled condition to further illustrate aspects of the system 10. In the embodiment shown, the medical device control handle 12 has a handle housing 13 formed from multiple handle housing portions, and in particular a first handle housing portion 13a and a second handle housing portion 13b. As will be discussed, the housing portions 13a and 13b are sized and have internal structures configured to fix the position of internal component(s) of the handle 12 relative to the handle housing 13 when attached together to form the handle housing 13. In this regard, in respect of the flush port/seal assembly 51 (see e.g. FIG. 4) including the connector cap 50, the flush port stem 30, the flush body portion 34, the proximal seal body portion 56, the seal element 62, and the seal cap 64, the handle housing portions 13a and 13b define respective inwardly-directed walls 80a and 80b that in the assembled handle 13 contact outer wall portions of seal cap 64 and hold seal cap 64 in a radially-secured position within the handle housing 13 spaced from outer walls of the housing 13 defined by the housing portions 13a and 13b. The walls 80a and 80b can in some aspects form a collar that surrounds outer wall portions of seal cap 64. As well, the housing portions 13a and 13b define respective inwardly-directed walls 82a and 82b that define an opening in the assembled handle 13 that is smaller than the greatest external dimension of cap 64 and that is positioned proximally of such greatest external dimension, so as to restrict proximal movement of the seal cap 64 and thereby the flush port/seal assembly 51. The inward edges of the walls 82a and 82b that define such opening can contact the cap 64 in the assembled handle housing 13 for these purposes. The handle housing portions 13a and 13b also define respective inwardly-directed walls 84a and 84b that in the assembled handle 13 have proximally-facing surface portions that contact distally-facing surface portions of the seal cap 64 and that secure seal cap 64 and thereby the flush port/seal assembly 51 against distal movement within the assembled handle 13. As illustrated, the walls 84a and 84b can also define an opening sized to contact and cradle external surfaces of the proximal seal body portion 56 to radially secure the position of proximal seal body portion 56 within the assembled handle 13. In some forms the walls 84a and 84b can form a collar in contact with and surrounding external surface of the proximal seal body portion 56. In the particular illustrated embodiment, walls 84a and 84b define a first opening within inward wall edges 86a and 86b that contact and cradle (e.g. form a collar that surrounds) a first, smaller-dimension segment 88 of the proximal seal body portion 56 and a second opening within inward wall edges 90a and 90b that contact and cradle (e.g. form a collar that surrounds) a second, larger-dimension mount segment 92 of proximal seal body portion 56. The larger-dimension mount segment 92 can in some forms have a non-circular external profile in cross-section, for example such external profile can be polygonal such as rectangular or square. In this manner, the engagement of inward wall edges 90a and 90b with surfaces of the non-circular external profile of the larger-dimension mount segment 92 can secure the flush port/seal assembly 51 against rotational movement relative to the handle housing 13. As well, walls 84a and 84b as shown include a first wall segment that extends generally along the longitudinal axis A-A and a second wall segment that extends perpendicular thereto (e.g. forming an “L” shape in cross-section), and together provide distally-facing surfaces that contact proximally-facing surface portions of larger-dimension mount segment 92 to secure the flush port/seal assembly against proximal movement relative to the handle housing 13, as well as proximally-facing surfaces that contact distally-facing surfaces of the shoulder 66 and/or of seal cap 64, so as to secure the flush port/seal assembly 51 against distal movement relative to the handle housing 13. In some forms, the flush port stem 30, the flush body portion 34, and the proximal seal body portion 56, can be subcomponents of a unitary, integral flush port/seal body member as illustrated for example in FIG. 4.

While certain embodiments and structures relating to the incorporation of internal walls of the handle housing 13 to secure the position of the flush port/seal assembly 51 have been discussed above, it will be understood that in broader aspects the present disclosure contemplates those discussed and/or other internal wall structures of the handle housing 13 that secure the assembly 51 in position relative to the handle housing 13, for example including securing the assembly 51 against proximal and/or distal movement relative to the handle housing 13, and/or against rotational movement relative to the handle housing 13, and/or against lateral movement relative to the handle housing 13 (e.g. maintaining components thereof radially secured at a consistent spaced distance from inner surfaces of laterally-adjacent outer walls the handle housing 13). This can be achieved for example by providing one or a plurality of inwardly-extending wall structure(s) of the handle housing 13 that contact external surfaces of the flush port/seal assembly 51 to secure the position thereof. Where the handle housing 13 includes multiple pieces (e.g. two, three or four pieces) that mate and are attached together to form the housing 13, inwardly extending wall structure(s) on each piece can coordinate to contact the external surfaces of the assembly 51 to provide the positional securement of the assembly 51 (e.g. securement against rotational, proximal, distal, and/or lateral movement) relative to the handle housing 13.

With particular reference to FIG. 5 along with FIGS. 1 and 2, the handle 12 also includes a locking member 100. The locking member 100 is connected to the handle housing 13 and movable between a locked position and unlocked position. When in the locked position, the locking member 100 may be in contact with the actuator button 20 and be configured to restrict the movement of the actuator button 20 along the longitudinal axis A-A. For example, in the illustrated embodiment, when the actuator button 20 is in a distal position at which the snare loop 16 (or other medical device) is positioned within the lumen 22 of the sheath 14, movement of the locking member 100 to the locked position can be achieved by pressing on trigger portion 102 (e.g. with an index finger) to pivot the locking member 100 in a first direction and drive the proximal end 104 into the interior of handle housing 13 to a position distal of and that interferes with distal movement of the actuator button 20. In some forms, such a locked position can include contact between the proximal end 104 of the locking member 100 and the actuator button 20 (e.g. with a distal face portion of actuator button 20). The handle housing 13 can, for example by contours of the housing portions 13a and 13b, define an opening in a location corresponding to all or a portion of the locking member 100, to allow travel of a portion of the locking member including the proximal end 104 into and out of the interior of the handle housing 13 for these purposes. To move the locking member 100 to the unlocked position from the locked position, a user can press on an unlocking portion 105 (e.g. with their index finger) to pivot the member 100 in a second direction opposite the first direction so as to drive the proximal end 104 of member 100 in a direction outward of the interior of the handle housing 13 to eliminate interference with the actuator button 20 and allow the actuator button 20 to be moved distally within the movement space defined by cutout 15, so as to deploy the snare loop 16 or other medical device structure out of the distal opening 24 of the lumen 22 of the sheath 14.

In the illustrated embodiment, to secure the locking member 100 to the housing 13, the locking member 100 defines laterally-extending posts 106 and 108 and the housing portions 13a and 13b respectively define corresponding openings 110 and 112 to receive the posts 106 and 108 when the portions 13a and 13b are attached together to form the housing 13. The posts 106 and 108 can rotate within the openings 110 and 112 to allow the locking member 100 to pivot in the first and second directions and between the locked and unlocked positions, as discussed above. Also in the embodiment shown, a spring 114 is included in the handle 12 with a first end positioned against the proximal surface 116 of locking member extension 118 and a second end captured in a cavity formed in the handle housing 13. For example, the handle housing may define a finger rest 117, for example in the form of a fin or other projecting member extending transverse to the longitudinal axis A-A, and in some forms the cavity in which the second end of spring 114 is captured can be defined within the fin or other finger rest 117, for instance by finger rest portions 117a and 117b of housing portions 13a and 13b. The spring 114 is positioned and configured to bias the locking member 100 in the locked position when forcibly moved from the unlocked position to the locked position and to bias the locking member 100 in the unlocked position when forcibly moved from the locked position to the unlocked position. The spring 114 can transition between alternately curved conditions when providing such biasing functions. The finger rest 117 in beneficial embodiments will occur on a side of the handle housing 13 that is opposite the side in which the cutout 15 is defined. In this manner, in a one-handed manual operation, a user can grip around the handle 12 and position a thumb of their hand on the actuator button 20 (e.g. a thumb wheel) with another finger (e.g. the index finger) of that hand positioned distal of and against the finger rest 117 and potentially still another finger (e.g. the middle finger) of that hand positioned proximal of and against finger rest 117, facilitating a stable grip on the handle 12 during proximal and distal movement of the actuator button 20 with the thumb. Also, when locking member 100 is included, such a hand position at the same time facilitates use of the index finger positioned distal of the finger rest 117 to press on locking member 100 to move it between its locked and unlocked positions, e.g. as discussed above. Also, the finger rest 117 in some embodiments can occur on the same side of the handle housing 13 from which flush port stem 30 extends, and potentially being generally aligned with finger rest 117 about the circumference of the handle 12 considered in its longitudinal profile.

As noted above, the housing 13 of the illustrated embodiment has two housing portions, 13a and 13b. The illustrated portions are generally in the form of coordinating housing shells that come against one another along contours of their outer edges. The housing portions 13a and 13b, or other housing portions of multiple-piece housings herein, can be secured to one another using any suitable technique or mechanism, including for example by correspondingly-located holes and pins defined by the respective housing portions, by adhesives or bonding agents, by welding (e.g. ultrasonic welding), or combinations thereof, or other means. These and other attachment techniques will be understood as suitable by skilled persons given the disclosures herein. In addition or alternatively, the housing portions 13a and 13b, or other housing portions of multiple-piece housings herein, can in some forms each be a unitary, integral piece, for example a unitary, integrally molded (e.g. injection molded) piece.

In other features, the housing 13 has a tapered distal cone portion 120 defined by hemi-cone portions 120a and 120b of housing portions 13a and 13b, respectively. In some embodiments as shown, a proximal region of the connector cap 50 extends out of and distally beyond the distalmost end of the cone portion 120. The handle housing 13, and in the shown embodiment the cone portion 120 thereof, also defines an opening 122 for receiving the flush port stem 30 such that stem 30 resides partially within and partially exterior of the handle housing 13. Opening 122 in the embodiment shown is provided by semi-circular cutouts 122a and 122b in housing portions 13a and 13b, respectively. Housing 13 also has interior surfaces that are complementary to the external profile of actuator button 20 so as to provide a longitudinally-extending slideway that guides the longitudinal movement of actuator button 20 relative to housing 13. Such interior surfaces for the slideway can include for example concavely-curved lateral surfaces 124a and 124b of housing portions 13a and 13b, respectively, that are complementary to convexly-curved outer surfaces on the actuator button 20. Housing portion 13a defines a plurality of holes 126a (for example defined by receptacle bosses) and housing portion 13b defines a plurality of correspondingly-located pins 126b that friction and/or snap fit within holes 126a to attach housing portions 13a and 13b to one another. Such attachments can be sufficient for the finished handle 12 or system 10, or other attachment techniques can be used instead of or in addition to the use of holes 126a and pins 126b.

The medical device control handles 12 and systems 10 incorporating them, including the various parts contained therein, may be formed from polymeric or plastic materials, ceramic materials, metals or metal alloys, and/or combinations thereof. The materials are selected so that they exhibit desirable or required performance characteristics, such as biocompatibility, flexibility, and strength to name a few. The polymeric or plastic materials may include one or more thermoplastic materials or thermoset materials, individually or in combination. Several examples of suitable polymeric or plastic materials may include but not limited to polyamides (e.g., nylons), polyimides, polyethylenes, polyurethanes, polyethers, polyesters, acrylonitrile butadiene styrene (ABS), and mixtures or copolymers thereof. In one form, the handle housing 13 of the handle 12 is formed from ABS. Any metal parts used within the system 10 may be formed from, without limitation, stainless steel, brass, a nickel-titanium allow such as nitinol, or a combination thereof.

In accordance with some aspects, the components described herein can be assembled in any suitable fashion or order to provide the system 10. However, in certain preferred forms, a core assembly will first be assembled that includes the snare loop 16 or other medical device structure, the sheath 14, the flush port/seal assembly 51, the actuator button 20, and the control shaft 18 attached to the snare loop 16 or other medical device and extending through the lumen 22 of the sheath 14 and through the flush port/seal assembly 51 and proximally attached to the actuator button 20. This core assembly can then be associated with the handle housing 13, for example by assembling multiple handle housing portions (e.g. housing portions 13a and 13b as discussed above) over and around the actuator button 20 and the flush port/seal assembly 51, and a segment of the control shaft 18 extending between the actuator button 20 and the flush port/seal assembly 51, and connecting such handle housing portions to one another e.g. as discussed herein. The assembly and connection of such multiple handle housing portions can beneficially secure the position of the flush port/seal assembly 51 relative to the handle housing 13 with inwardly-directed wall structures as discussed hereinabove. These assembly operations, and the system 10 component configurations that facilitate them, provide for a particularly convenient and practicable manufacture of the system 10. Also, skilled persons will understand that where locking member 100 and spring 114 are included, these can be placed and captured between the multiple handle housing portions (e.g. 13a and 13b) as such portions are assembled together and connected to one another, to provide their positions and functions as described hereinabove.

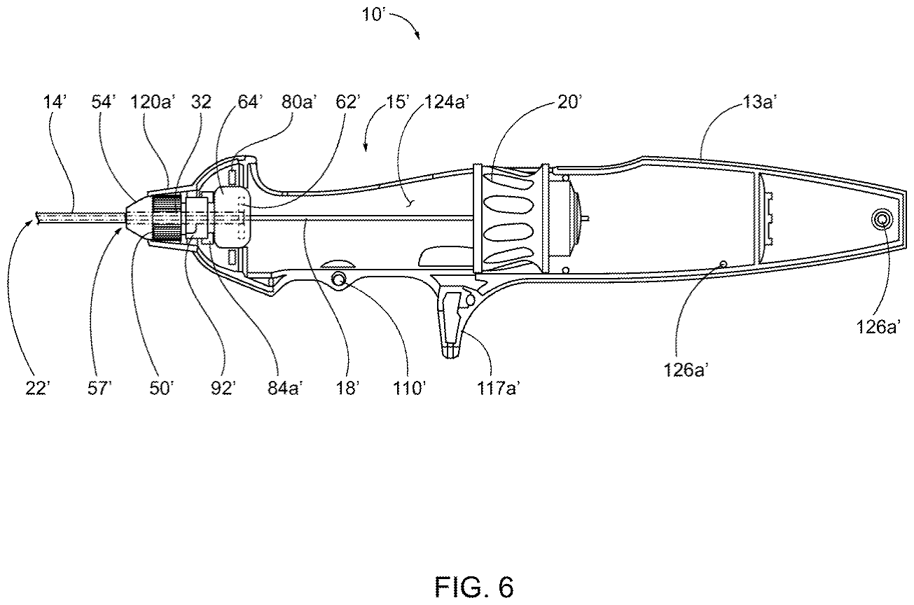

Referring now to FIG. 6, shown are components of an alternate medical device control system 10′ that can have components similar to those in system 10 discussed above which are similarly numbered in FIG. 6 along with a prime symbol “′”, except system 10′ is equipped with a seal assembly but does not include a flush port assembly integral with the seal assembly. It will be understood that system 10′ will include a second handle housing portion (not shown) correspondingly shaped to and that is attached to handle housing portion 13a′ in forming the handle of system 10′, similar to housing portions 13a and 13b as discussed in conjunction with system 10 above. As shown in FIG. 6, the seal assembly of system 10′ includes a seal body portion 56′ that defines an internal lumen 58′, a seal element 62′ such as a seal disc, and a seal cap 64′ attached, for example press fitted, to the seal body portion 56′ and pressing the seal element against the seal body portion 56′, potentially compressing the seal element against the seal body portion 56′. The seal assembly of system 10′ is similarly positionally secured within the medical device control handle of system 10′ by internal wall structures of the handle housing, for example walls 80a′ and/or 84a′ and corresponding inwardly-directed walls on the included second handle housing portion, as discussed above in conjunction with system 10 and its corresponding inwardly-directed support wall structures. In system 10′, the housing portion 13a′ can define a further inwardly-directed wall 85′ positioned distal of enlarged seal body portion 92′. Wall 85′ (along with a corresponding inwardly-directed wall on the included second handle housing portion) can abut a distal face of the enlarged seal body mount portion 92′ to prevent distal movement of the seal assembly and/or can contact and cradle (e.g. to form a collar that surrounds) correspondingly-located external surfaces of the seal body portion 56′ to hold the seal assembly in a radially-secured position within the handle housing spaced from outer walls of the housing.

When system 10 or 10′ is equipped with a snare capture element such as a snare loop 16, system 10 or 10′ can be used in the retrieval of a vascular filter implanted in the vasculature of a patient, for example a vena cava filter implanted in a vena cava of a patient. It will be understood, however, that other uses of systems in accordance with the present disclosure, including those equipped with a snare loop, may be made. Generally in such uses, the sheath of the system 10 or 10′ (potentially after flushing the lumen of the sheath for example as described above when a flush port is included as in system 10), can be advanced into the vasculature of a patient. The actuator button 20 or 20′ can be manipulated by hand by the user to advance and/or withdraw and/or rotate the control shaft 18 or 18′, which in turn may manipulate (e.g. including deploy) a medical device structure in the vasculature of the patient, so as to treat the patient. These as well as other operations or uses of the systems 10 or 10′ as described herein will be apparent to those skilled in the art and are contemplated in aspects of the present disclosure.

Medical device control systems 10 or 10′ or medical device control handles as described herein can be sterilized for use. For example they can be packaged in a medical package defining a sterile barrier within which they are positioned, and the package with the system 10 or 10′ or the medical device control handle can be terminally sterilized using a suitable technique such as exposure to ethylene oxide gas or radiation.

The uses of the terms “a” and “an” and “the” and similar references herein (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected.

1. A system for hand-held control of a medical device structure, comprising:
a control handle including a handle housing and an actuator movably mounted to the handle housing and arranged for manual operation by a hand of a user;
a control shaft connected to the actuator;
a sheath attached to a distal end of the handle housing and having a lumen extending to a distal opening of the sheath, with the control shaft extending from the handle housing and into the lumen of the sheath;
a medical device structure attached to the control shaft, with the actuator being operable by the hand of the user to cause movement of the control shaft distally relative to the handle housing so as to deploy the medical device structure from the distal opening of the sheath and proximally relative to the handle housing so as to retract the medical device structure into the distal opening of the sheath;
a seal element mounted in the handle housing, with the control shaft extending through the seal element, wherein the seal element is cooperable with the control shaft to maintain a seal against the control shaft during said movement of the control shaft; and
a flush port opening in fluid communication with the lumen of the sheath, optionally wherein the flush port opening is on the control handle.

2. The system of claim 1, wherein the seal element sealingly interrupts a fluid passage extending from the distal opening of the sheath, through the lumen of the sheath and into an interior space of the handle housing.

3. The system of claim 1, wherein the seal element comprises at least a first opening therein, with said control shaft received through said first opening and being longitudinally slidable and rotatable relative to said first opening.

4. The system of claim 3, wherein the seal element comprises at least a first elastomeric disc.

5. The system of claim 1, comprising at least one of, or both of:
a. a seal assembly mounted within the handle housing, the seal assembly including the seal element received against a seal body portion defining a first lumen, the seal assembly optionally also including a seal cap attached to the seal body portion and pressing the seal element against the seal body portion; and
b. a flush port assembly mounted within the handle housing, the flush port assembly including a flush port body including a first flush port body portion defining a first flush lumen portion and a second flush port body portion extending transversely to the first flush port body portion and defining a second flush lumen portion extending from the flush port opening and intersecting and fluidly communicating with the first flush lumen portion, wherein the second flush port body portion provides a flush port stem.

6. The system of claim 5, wherein:
a. the handle housing has one or more inwardly-directed walls that contact and positionally fix the seal assembly relative to the handle housing, optionally wherein the one or more inwardly-directed walls contact and positionally fix the seal assembly against distal, proximal, lateral and rotational movement relative to the handle housing;
and/or
b. the handle housing has one or more inwardly-directed walls that contact and positionally fix the flush port assembly relative to the handle housing, optionally wherein the one or more inwardly-directed walls contact and positionally fix the flush port assembly against distal, proximal, lateral and rotational movement relative to the handle housing.

7-8. (canceled)

9. The system of claim 5, comprising both the seal assembly and the flush port assembly.

10. The system of claim 9, wherein the flush port assembly and the seal assembly are subcomponents of a flush port/seal assembly.

11. The system of claim 10, wherein the seal body portion and the flush port body are segments of an integral flush port/seal body.

12. The system of claim 10, wherein the handle housing has one or more inwardly-directed walls that contact and positionally fix the flush port/seal assembly relative to the handle housing, optionally wherein the one or more inwardly-directed walls contact and positionally fix the flush port/seal assembly against distal, proximal, lateral and rotational movement relative to the handle housing.

13. The system of claim 12, wherein the handle housing is composed of at least a first handle housing portion and a second handle housing portion connected together, wherein the first handle housing portion includes a first inwardly-directed wall portion and the second handle housing portion includes a second inwardly-directed wall portion, and wherein the first and second inwardly-directed wall portions align with one another and have respective inward end surfaces that contact and cradle an exterior surface of the flush port/seal assembly.

14-21. (canceled)

22. The system of claim 1, wherein the actuator is also rotatable by the hand of the user to cause rotation of the control shaft relative to the handle housing so as to rotate the medical device structure.

23-25. (canceled)

26. The system of claim 1, wherein the medical device structure is a vascular snare element.

27-34. (canceled)

35. A method for preparing a system prior to use on a patient, comprising:
providing a system according to claim 1; and
passing a flush liquid into the flush port opening, through the lumen of the sheath, and out the distal opening of the sheath.

36. A method for treating a patient using a medical device, comprising:
providing a system according to claim 1;
passing a flush liquid into the flush port, through the lumen of the sheath, and out the distal opening of the sheath;
after said passing a flush liquid, introducing the sheath into a vascular vessel of the patient;
operating the actuator by hand to cause movement of the control shaft distally relative to the handle housing so as to deploy the medical device structure from the distal opening of the sheath; and
treating the patient with the medical device structure.

37. The method of claim 36, wherein the medical device structure is a vascular snare element, and wherein the treating comprises securing and removing an implant from the vascular vessel with the vascular snare element.

38-39. (canceled)

40. A medical device control handle for hand-held control of a medical device structure that is connected to a control shaft and deployable from a lumen of a sheath, the medical device control handle comprising:
a handle housing;
an actuator movable relative to the handle housing and arranged for manual operation by a hand of a user, the actuator being connectable to the control shaft; and
at least one of, or both of:
a. a seal assembly mounted in the handle housing and configured to maintain a seal against an outer surface of a control shaft for a medical device, the seal assembly including a seal body defining a lumen therein and a seal element received against the seal body; and
b. a flush port assembly mounted in the handle housing, the flush port assembly including a flush port body having a first flush port body portion defining a first flush lumen portion therein configured to fluidly communicate with a sheath lumen and a second flush port body portion extending transverse to the first flush port body portion and providing a flush port stem defining a second flush lumen portion therein and extending to a flush port opening, the second flush lumen portion intersecting and fluidly communicating with the first flush lumen portion.

41. The medical device control handle of claim 40, including the seal assembly, the seal assembly optionally also including a seal cap attached to the seal body so as to press the seal element against the seal body portion.

42. The medical device control handle of claim 40, including the flush port assembly.

43. The medical device control handle of claim 40, including both the seal assembly and the flush port assembly.

44. The medical device control handle of claim 42, wherein the handle housing has one or more inwardly-directed walls that contact and positionally fix the flush port assembly when present or the seal assembly when present relative to the handle housing.

45. The medical device control handle of claim 40, including both the seal assembly and the flush port assembly, and wherein the flush port assembly and the seal assembly are subcomponents of a flush port/seal assembly.

46. (canceled)

47. The medical device control handle of claim 45, wherein the handle housing has one or more inwardly-directed walls that contact and positionally fix the flush port/seal assembly relative to the handle housing, and wherein the one or more inwardly-directed walls contact and positionally fix the flush port/seal assembly against distal, proximal, lateral and rotational movement relative to the handle housing.

48-58. (canceled)

59. A medical device control system including a medical device control handle according to claim 40, the sheath, the control shaft, and the medical device structure attached to the control shaft.

60. (canceled)
